(19) United States
(12) Patent Application Publication
(10) Pub. No.: US 2006/0058609 A1
(43) Pub. Date: Mar. 16, 2006

(54) Extracting ultrasound summary information useful for inexperienced users of ultrasound

(75) Inventor: Bjorn Olstad, Stathelle (NO)

Correspondence Address: MCANDREWS HELD & MALLOY, LTD, 500 WEST MADISON STREET, SUITE 3400, CHICAGO, IL, 60661, US

(73) Assignee: General Electric Company

(21) Appl. No.: 11/082,540

(22) Filed: Mar. 17, 2005

Related U.S. Application Data
(60) Provisional application No. 60/605,939, filed on Aug. 31, 2004.

(51) Int. Cl. A61B 5/05

(52) U.S. Cl. 600/407

(57) ABSTRACT

The present invention relates to a method and apparatus for generating an image responsive to moving cardiac structure and blood, and extracting clinically relevant information based on anatomical landmarks located within the heart. One embodiment of the present invention comprises at least one processor responsive to signals received from the heart used to acquire an apical view of the heart, generate an image of the apical view on a display, automatically identify an AV-plane of the heart and generate a clinical executive report using the identified AV-plane.

### RELATED APPLICATIONS/INCORPORATION BY REFERENCE

This application is related to, and claims benefit of and priority from, Provisional Application No. 60/605,939, filed Aug. 31, 2004, titled “EXTRACTING ULTRASOUND SUMMARY INFORMATION USEFUL FOR INEXPERIENCED USERS OF ULTRASOUND”, the complete subject matter of which is incorporated herein by reference in its entirety.

The complete subject matter of each of the following U.S. Patent Applications is incorporated by reference herein in their entirety:
U.S. patent application Ser. No. 10/248,090 filed on Dec. 17, 2002.
U.S. patent application Ser. No. 10/064,032 filed on Jun. 4, 2002.
U.S. patent application Ser. No. 10/064,083 filed on Jun. 10, 2002.
U.S. patent application Ser. No. 10/064,033 filed on Jun. 4, 2002.
U.S. patent application Ser. No. 10/064,084 filed on Jun. 10, 2002.
U.S. patent application Ser. No. 10/064,085 filed on Jun. 10, 2002.

### FEDERALLY SPONSORED RESEARCH OR DEVELOPMENT

[Not Applicable]

### BACKGROUND OF THE INVENTION

Embodiments of the present invention relate to an ultrasound system. More specifically, embodiments of the present invention relate to an ultrasound system for imaging a heart and extracting clinically relevant information from the heart.

Echocardiography is a branch of the ultrasound field that is currently a mixture of subjective image assessment and extraction of key quantitative parameters. In the past, evaluating cardiac function has been hampered by a lack of well-established parameters used to increase the accuracy and objectivity in the assessment of diseases (coronary artery diseases for example). It has been shown that inter-observer variability between echo-centers is unacceptably high due to the subjective nature of the cardiac motion assessment.

Technical and clinical research has focused on this problem, aimed at defining and validating quantitative parameters. Encouraging clinical validation studies have been reported indicating a set of new potential parameters that may be used to increase objectivity and accuracy in the diagnosis of, for instance, coronary artery diseases. Many of the new parameters are difficult or impossible to assess directly by visual inspection of the ultrasound images generated in real-time. The quantification has typically required a post-processing step with tedious, manual analysis to extract the necessary parameters. Determination of the location of anatomical landmarks in the heart is no exception. Time intensive post-processing techniques or complex, computation-intensive real-time techniques are undesirable.

One method disclosed in U.S. Pat. No. 5,601,084 to Sheehan et al. describes imaging and three-dimensionally modeling portions of the heart using imaging data. Another method disclosed in U.S. Pat. No. 6,099,471 to Torp et al. describes calculating and displaying strain velocity in real time. Still another method disclosed in U.S. Pat. No. 5,515,856 to Olstad et al. describes generating anatomical M-mode displays for investigations of living biological structures, such as heart function, during movement of the structure. Yet another method disclosed in U.S. Pat. No. 6,019,724 to Gronningsaeter et al. describes generating quasi-real-time feedback for the purpose of guiding procedures by means of ultrasound imaging.

Ultrasound devices are used to conduct subjective assessment of the cardiac wall function. Such subjective assessment requires extensive training, especially in emergency situations. This thus necessarily limits the potential user's ability to perform meaningful cardiac examinations. One or more embodiments of the present invention enable users (including inexperienced users such as emergency personnel and private physicians for example) to use an ultrasound device (a hand-held device for example) to perform meaningful cardiac examinations and extract summary information.

### BRIEF SUMMARY OF THE INVENTION

An embodiment of the present invention relates to an ultrasound system for imaging a heart and extracting clinically relevant information from the heart. More specifically, an embodiment of the present invention relates to an ultrasound system for imaging a heart and extracting clinically relevant information from the heart. after automatically locating anatomical landmarks within the heart.

One embodiment of the present invention relates to a system and measure for generating an image responsive to moving cardiac structure and blood. One or more embodiments of the present invention enables users (including inexperienced users such as emergency personnel and private physicians for example) to use an ultrasound device (a hand-held device for example) to perform meaningful cardiac examinations and extract summary information.

An apparatus is provided in an ultrasound machine for imaging a heart and extracting certain clinically relevant information from the heart based on having previously located certain anatomical landmarks within the heart. In such an environment, an apparatus for extracting the clinically relevant information comprises a front-end arranged to transmit ultrasound waves into a structure and to generate received signals in response to ultrasound waves backscattered from said structure over a time period. A processor responsive to the received signals generates a set of analytic parameter values representing movement of the cardiac structure over the time period and analyzes elements of the set of analytic parameter values to automatically extract position information of the anatomical landmarks and track the positions of the landmarks. A processor responsive to the tracked anatomical landmark positions extracts certain clinically relevant information from certain locations within the heart with respect to the tracked anatomical landmarks. A display is arranged to overlay indicia corresponding to the position information onto an image of the moving structure, indicating to an operator the position of the tracked anatomical landmarks and displaying the extracted clinically relevant information. A method is also provided in an ultrasound machine for imaging a heart and extracting certain clinically relevant information from the heart based on having previously located certain anatomical landmarks within the heart. In such an environment a method for extracting the clinically relevant information comprises transmitting ultrasound waves into a structure and generating received signals in response to ultrasound waves backscattered from the structure over a time period. A set of analytic parameter values is generated in response to the received signals representing movement of the cardiac structure over the time period. Position information of the anatomical landmarks is automatically extracted and the positions of the landmarks are then tracked. Certain clinically relevant information is extracted from certain locations within the heart with respect to the tracked anatomical landmarks. Indicia corresponding to the position information are overlaid onto the image of the moving structure to indicate to an operator the position of the tracked anatomical landmarks and the extracted clinically relevant information is also displayed. In at least one embodiment, the clinical executive report comprises at least one of the following parameters: Ejection Fraction, AV-motion, Heart Rate, sinus rhythm, contractions, mitral flow and detected arrhythmias.

Certain embodiments of the present invention afford an approach to extract certain clinically relevant information from a heart after automatically locating key anatomical landmarks of the heart, such as the apex and the AV-plane.

### BRIEF DESCRIPTION OF THE DRAWINGS

FIG. 1 depicts a block diagram of an embodiment of an ultrasound machine made in accordance with various embodiments of the present invention.

FIG. 2A depicts a schematic block diagram of a portable diagnostic ultrasound system formed in accordance with an embodiment of the present invention such that digital beamforming is performed within a hand-held probe assembly.

FIG. 2B depicts a realistic illustration of the portable diagnostic ultrasound system of FIG. 2A in accordance with various embodiments of the present invention.

FIGS. 3A and 3B depict flowcharts illustrating an embodiment of a method performed by the machine shown in FIG. 1, in accordance with various embodiments of the present invention.

FIG. 4 illustrates using the methods of FIGS. 3A and 3B to generate one or more clinical executive reports in accordance with an embodiment of the present invention.

FIGS. 5A and 5B depict examples of ECGs of normal sinus rhythms.

FIG. 5C depicts an example of an ECG of a supraventricular tachycardia.

FIG. 5D depicts an example of an ECG of an atrial flutter.

FIG. 5E depicts an example of an ECG of a ventricular tachycardia.

FIG. 5F depicts an example of an ECG of an atrioventricular block.

FIG. 5G depicts an example of an ECG of a complete AV block.

FIG. 5H depicts an example of an ECG of a premature atrial contraction.

FIG. 5I depicts an example of an ECG of a premature ventricular contraction.

FIG. 5J depicts an example of an ECG of an atrial fibrillation.

The foregoing summary, as well as the following detailed description of certain embodiments of the present invention, will be better understood when read in conjunction with the appended drawings. It should be understood, however, that the present invention is not limited to the arrangements and instrumentality shown in the attached drawings.

### DETAILED DESCRIPTION OF THE INVENTION

An embodiment of the present invention enables the real-time extraction of clinically relevant information. Another embodiment of the present invention enables the real-time extraction of clinically relevant information from within a heart after locating and tracking certain anatomical landmarks of the heart. Moving cardiac structure is monitored to accomplish this function. As used herein, the term structure comprises non-liquid and non-gas matter, such as cardiac tissue for example. An embodiment of the present invention provides improved, real-time visualization and assessment of certain clinically relevant parameters of the heart. The moving structure is characterized by a set of analytic parameter values corresponding to anatomical points within a myocardial segment of the heart. The set of analytic parameter values may comprise, for example, tissue velocity values, time-integrated tissue velocity values, B-mode tissue intensity values, tissue strain rate values, blood flow values, and mitral valve inferred values.

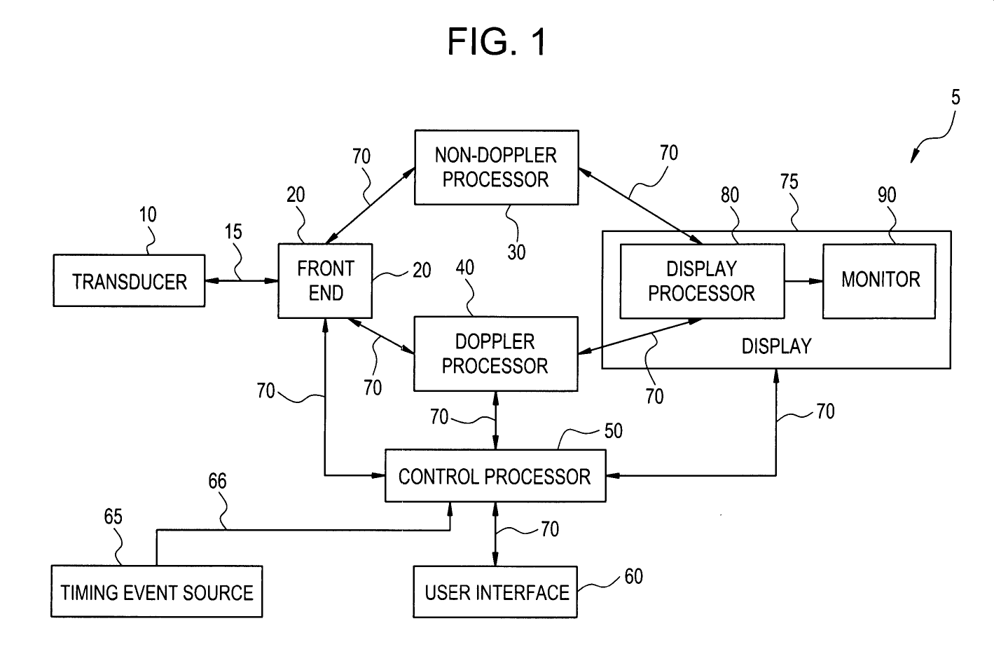

FIG. 1 illustrates an embodiment of an ultrasound machine, generally designated 5, in accordance with embodiments of the present invention. A transducer 10 transmits ultrasound waves into a subject by converting electrical analog signals to ultrasonic energy and receives the ultrasound waves backscattered from the subject by converting ultrasonic energy to analog electrical signals. A front-end 20, that in one embodiment comprises a receiver, transmitter, and beamformer, may be used to create the necessary transmitted waveforms, beam patterns, receiver filtering techniques, and demodulation schemes that are used for the various imaging modes. Front-end 20 performs such functions, converting digital data to analog data and vice versa. Front-end 20 interfaces to transducer 10 using analog interface 15 and interfaces to a non-Doppler processor 30, a Doppler processor 40 and a control processor 50 over a bus 70 (digital bus for example). Bus 70 may comprise several digital sub-buses, each sub-bus having its own unique configuration and providing digital data interfaces to various parts of the ultrasound machine 5.

Non-Doppler processor 30 is, in one embodiment, adapted to provide amplitude detection functions and data compression functions used for imaging modes such as B-mode, M-mode, and harmonic imaging. Doppler processor 40, in one embodiment provides clutter filtering functions and movement parameter estimation functions used for imaging modes such as tissue velocity imaging (TVI), strain rate imaging (SRI), and color M-mode. In one embodiment, the two processors, 30 and 40, accept digital signal data from the front-end 20, process the digital signal data into estimated parameter values, and pass the estimated parameter values to processor 50 and a display 75 over digital bus 70. The estimated parameter values may be created using the received signals in frequency bands centered at the fundamental, harmonics, or sub-harmonics of the transmitted signals in a manner known to those skilled in the art.

Display 75 is adapted, in one embodiment, to provide scan-conversion functions, color mapping functions, and tissue/flow arbitration functions, performed by a display processor 80 which accepts digital parameter values from processors 30, 40, and 50, processes, maps, and formats the digital data for display, converts the digital display data to analog display signals, and communicate the analog display signals to a monitor 90. Monitor 90 accepts the analog display signals from display processor 80 and displays the resultant image.

A user interface 60 enables user commands to be input by the operator to the ultrasound machine 5 through control processor 50. User interface 60 may comprise a keyboard, mouse, switches, knobs, buttons, track balls, foot pedals, voice control and on-screen menus, among other devices.

A timing event source 65 generates a cardiac timing event signal 66 that represents the cardiac waveform of the subject. The timing event signal 66 is input to ultrasound machine 5 through control processor 50.

In one embodiment, control processor 50 comprises the central processor of the ultrasound machine 5, interfacing to various other parts of the ultrasound machine 5 through digital bus 70. Control processor 50 executes the various data algorithms and functions for the various imaging and diagnostic modes. Digital data and commands may be communicated between control processor 50 and other various parts of the ultrasound machine 5. As an alternative, the functions performed by control processor 50 may be performed by multiple processors, or may be integrated into processors 30, 40, or 80, or any combination thereof. As a further alternative, the functions of processors 30, 40, 50, and 80 may be integrated into a single PC backend.

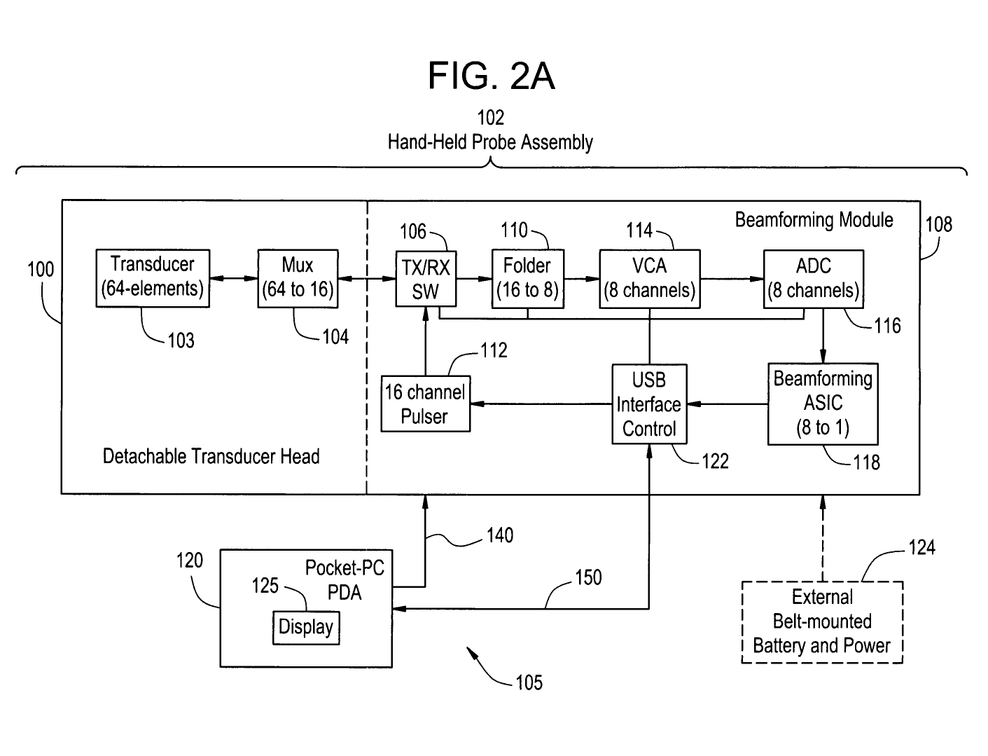

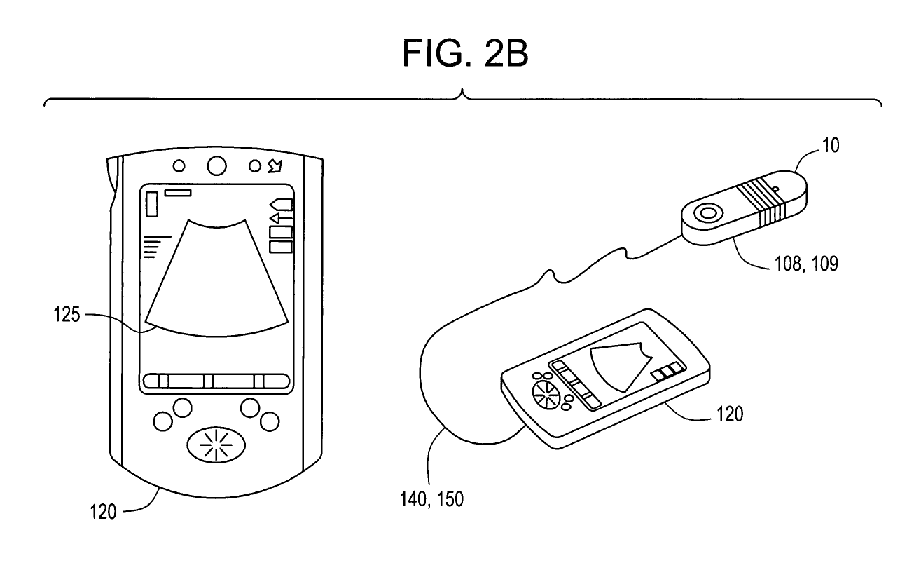

FIG. 2A depicts a schematic block diagram of a portable ultrasound system 105 in accordance with at least one embodiment of the present invention. Certain embodiments of the ultrasound system 105 may comprise a detachable transducer module 100, a beamforming module 108, a PDA device 120, and, optionally, an external battery/power source 124. The transducer module 100 attaches to the beamforming module 108 to forming a hand-held probe assembly 102. In an embodiment of the present invention, the PDA 120 includes an internal battery to power the PDA 120 and the hand-held probe assembly 102. A battery power interface 140 connects between the PDA 120 and the hand-held probe assembly 102. FIG. 2B depicts a more realistic illustration of the ultrasound system 105.

The transducer module 100 comprises a 64-element transducer array 103 and a 64 channel to 16 channel multiplexer 104. The beamforming module 108 comprises a pulser 112, a TX/RX switching module 106, a folder module 110, a voltage controlled amplifier (VCA) 114, an analog-to-digital converter (ADC) 116, a beamforming ASIC 118, and a PDA interface controller 122. The PDA device 120 is a standard, off-the-shelf device such as a Palm Pilot running Windows applications such as Windows-CE applications and having a touch-screen display 125. The PDA 120 may be modified to include ultrasound data processing and application software to support a plurality of ultrasound imaging modes.

In the transducer module 100, the transducer array 103 is connected to the multiplexer 104. When the transducer module 100 is connected to the beamforming module 108, the multiplexer 104 is connected to an input of TX/RX switching module 106.

In the beamforming module 108, the output of the TX/RX switching module 106 connects to the input of the folder module 110 and the output of the folder module 110 connects to the input of the VCA 114. The output of the VCA 114 connects to the input of the ADC 116. The output of the ADC 116 connects to the input of the beamforming ASIC 118. The output of the beamforming ASIC 118 connects to the input of the PDA interface controller 122. The output of the 16-channel pulser 112 connects to an input of TX/RX switching module 106. Optionally, an external battery/power source 124 connects to beamforming module 108.

The PDA interface controller 122 connects to the pulser 112, and to the PDA device 120 through a standard digital interface 150. In an embodiment of the present invention, the standard digital interface 150 is a Universal Serial Bus (USB) interface and the PDA interface controller 122 is a USB controller. Optionally, the standard digital interface 150 may be a parallel interface where the PDA interface controller 122 is a PC card. Alternatively, the standard digital interface may be a wireless interface (Bluetooth for example) providing RF communication between the PDA interface controller 122 and the PDA 120.

The various elements of the portable ultrasound system 105 may be combined or separated according to various embodiments of the present invention. For example, the folder 110 and VCA 114 may be combined into a single processing element. Also, the external battery 124 may be integrated into the beamforming module 108, becoming an internal battery.

It is contemplated that one function of the PDA-based ultrasound scanner 105 (and the ultrasound machine 5) is to transmit ultrasound energy into a subject to be imaged, and receive and process backscattered ultrasound signals from the subject to create and display an image on the display 125 of the PDA device 120. A user selects a transducer head 100 to connect to the beamforming module 108 to form a hand-held probe assembly 102 to be used for a particular scanning application. The transducer head is selected from a group of transducers including linear arrays, curved arrays, and phased arrays. An imaging mode may be selected from a menu on the display 125 of the PDA device 120 using a touch-screen stylus.

To generate a transmitted beam of ultrasound energy, the PDA device 120 sends digital control signals to the PDA interface controller 122 within the beamforming module 108 through the standard digital interface 150. The digital control signals instruct the beamforming module 108 to generate transmit parameters to create a beam of a certain shape that originates from a certain point at the surface of the transducer array 103. The transmit parameters are selected in the pulser 112 in response to the digital control signals from the PDA device 120. The pulser 112 uses the transmit parameters to properly encode transmit signals to be sent to the transducer array 103 through the TX/RX switching module 106 and the multiplexer 104. The transmit signals are set at certain levels and phases with respect to each other and are provided to individual transducer elements of the transducer array 103. The transmit signals excite the transducer elements of the transducer array 103 to emit ultrasound waves with the same phase and level relationships as the transmit signals. As a result, a transmitted beam of ultrasound energy is formed in a subject within a scan plane along a scan line when the transducer array 103 is acoustically coupled to the subject by using, for example, ultrasound gel.

Once certain anatomical landmarks of the heart are identified, (e.g., the AV-planes and apex as described in U.S. patent application Ser. No. 10/248,090 filed on Dec. 17, 2002) certain clinically relevant information may be extracted and displayed to a user of the ultrasound system 5 or 105 in accordance with various aspects of the present invention. The various processors of the ultrasound machine 5 and 105 described above may be used to extract and display clinically relevant information from various locations within the heart.

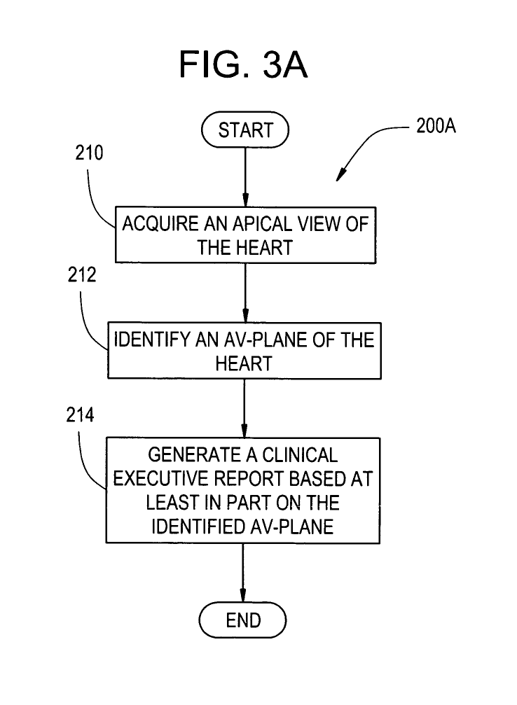

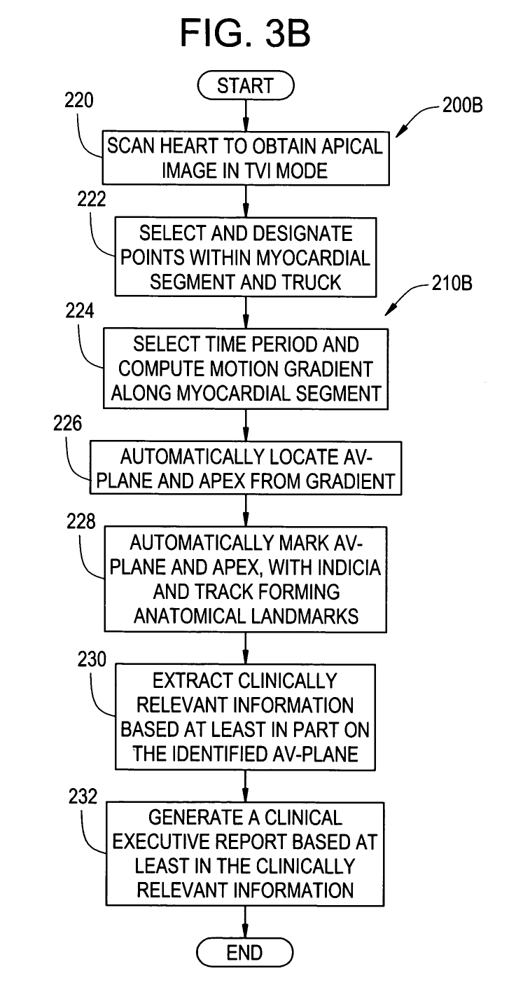

One embodiment of the present invention comprises a method of extracting clinical relevant information from clinically relevant locations. FIG. 3A depicts a high level flow chart illustrating a method 200A for generating a clinical executive report in accordance with various aspects of the present invention. In the illustrated embodiment, the method 200A comprises Step 210, which comprises acquiring an apical view of the heart while imaging the heart using ultrasound system 5 or 105 for example. In one embodiment, the image of the apical view is generated on display. Step 212 comprises identifying (automatically for example) an AV-plane of the heart, using at least in part, the acquired apical view. Step 214 comprises generating a clinical executive report based, at least in part, on the identified AV-plane.

FIG. 2B depicts a flow chart illustrating an embodiment of a method 200B (similar to method 200A depicted in FIG. 2A) performed using machines 5 or 105 illustrated in FIGS. 1, 2A and 2B for example in accordance with various aspects of the present invention. Method 200B comprises Step 220, scanning the heart to obtain one or more apical images in TVI mode. Step 222 comprises selecting and designating points within the myocardial segment and tracking.

One embodiment of method 200B may further comprise Step 224, selecting a time period and computing one or more motion gradients along at least one myocardial segment. Step 226 comprises automatically locating the AV-plane and apex using the gradient computed in Step 224 for example. Step 226 comprises automatically marking the AV-plane and apex with indicia and tracking, forming at least one anatomical landmark.

Method 200B may further comprise Step 230, comprises extracting clinically relevant information from, at least in part, the identified AV-plane (the at least one anatomical landmark). Step 232 comprises generating a clinical executive report based at least in part on the clinically relevant information.

As defined herein, clinically relevant information comprises at least one of Doppler profile information (i.e., over time), velocity profile information, strain rate profile information, strain profile information, M-mode information, deformation information, displacement information, and B-mode information although other clinically relevant information is contemplated.

One embodiment of the present invention relates to a system and measure for generating an image responsive to moving cardiac structure and blood. One or more embodiments of the present invention enable users (including inexperienced users such as emergency personnel and private physicians for example) to use an ultrasound device (a hand-held device for example) to perform meaningful cardiac examinations and extract and in at least one embodiment display summary information.

It should be appreciated that the heart essentially functions as an electromechanical pump. Each beat comprises two main actions: a synchronous contraction of the two upper chambers of the heart (the atria) drives blood into the lower chambers (the ventricles); and a synchronous contraction of the ventricles then ejects the blood into the circulatory system.

The rhythmic contractions of the heart are triggered by waves of electrical activity that spread from the sino-atrial node throughout the heart muscle. However, even the resting heart rate is not strictly periodic. There are small fluctuations in the time intervals between beats that are fractal in nature, and a loss in this variability is a sign of cardiac ill health.

However, a cardiac arrhythmia, in which the rhythm of electrical waves that drives the heart is broken can be lethal. A loss in the synchronized rhythm of the heart may cause different parts of the atrial or ventricular muscle to contract at different times, undermining the pumping action of the heart. An arrhythmia therefore leads to the mechanical failure of the heart.

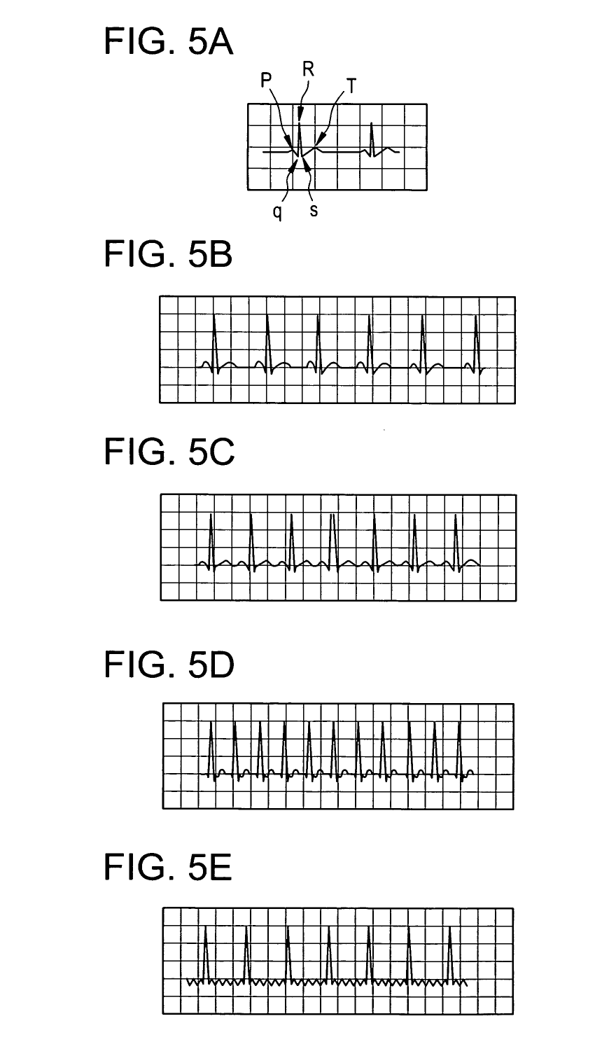

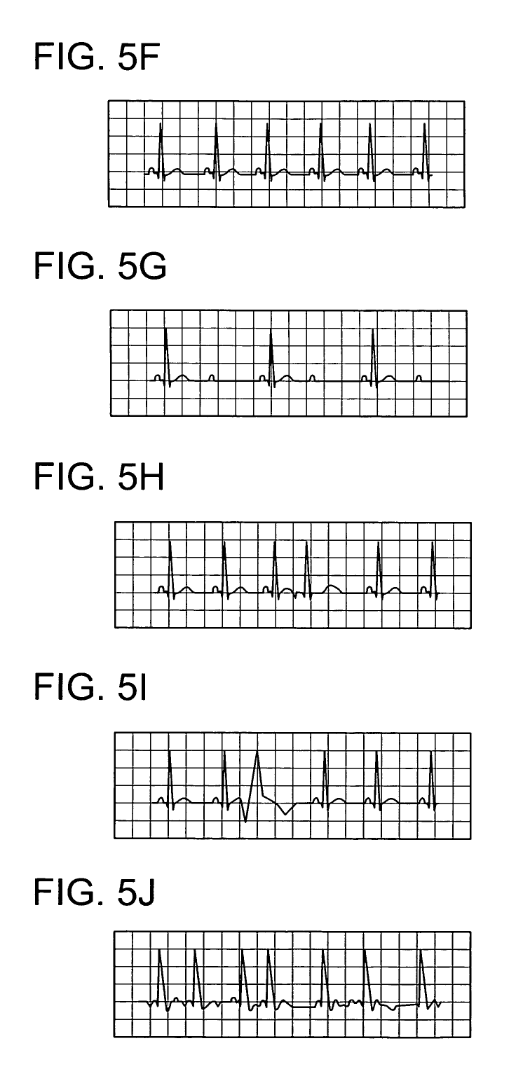

In a normal heart rhythm, the sinus node generates an electrical impulse which travels through the right and left atrial muscles producing electrical changes, represented on the electrocardiogram (ECG) by the p-wave as illustrated in FIG. 5A. The electrical impulse travels through the atrioventricular node, which conducts electricity at a slower pace. This creates a pause (a PR interval) before the ventricles are stimulated. This pause allows blood to be emptied into the ventricles prior to ventricular contraction. The ventricular contraction is represented electrically on the ECG by the QRS complex of waves. The following T-wave represents the electrical changes in the ventricles as they are relaxing.

Therefore, in an ECG with normal sinus rhythm, p-waves are followed after a brief pause by a QRS complex, then a T-wave as illustrated in FIG. 5A. The cycle repeats itself as depicted in FIG. 5B. Normal sinus rhythm not only indicates that the rhythm is normally generated and traveling in a normal fashion, but also that the heart rate is within normal limits.

It is contemplated that cardiac arrhythmias may comprise fast heart rates or tachycardias, slow heart rates and irregular heart rates. A fast heart rate may occur with a normal heart rhythm called sinus tachycardia. This means that the impulse generating the heart beats is normal, but they are occurring at a faster pace than normal.

Supraventricular tachycardia (SVT) is an abnormal heart rhythm wherein the impulse stimulating the heart is not generated by the sinus node, but instead is generated by collection of tissue around the AV node. These electrical impulses from this abnormal site are generated at a rapid impulse, which may reach 280 beats per minute as illustrated in FIG. 5C.

Atrial flutter comprises an abnormal rapid heart rhythm wherein the abnormal tissue generating the rapid heart rate is in the atria, however, the AV node is not involved. Since the AV node is slow conduction tissue, but is not involved in this type of abnormal heart rhythm, the heart rate in this case would be faster than that in supraventricular tachycardia where the AV node generates the abnormal heart rhythm causing it to be slower as illustrated in FIG. 5D.

Ventricular tachycardia comprises a dangerous type of rapid heart rhythm as it is usually associated with poor cardiac output (amount of blood ejected out of the heart). It results from abnormal tissues in the ventricles generating a rapid and irregular heart rhythm as illustrated in FIG. 5E.

A condition in which the heart slows down, yet maintains the normal patter of rhythm (sinus), is known as sinus bradycardia. It usually is benign and may be caused by medications such as beta blockers. One example of a slow heart rate is antrioventricular block (AVB). AVB may exist where the sinus node generates heart beats causing the atria to contract at a normal rate, however not every electrical impulse is being passed down to the ventricles due to a block in conduction. An example of an ECG of AVB is illustrated in FIG. 5F. It should be appreciated that there are various types of AV block depending upon the mechanism of block. Second degree AV block is when the impulse from the atria is blocked every certain number of beats. In complete AV block none of the atrial impulses pass through the atrioventricular node and the ventricles generate their own rhythm as illustrated in FIG. 5G.

An example of an irregular heart rhythm is referred to as premature atrial contraction (PAC). In PAC, the atria fires an early impulse which causes the heart to beat earlier causing irregularity in the heart rhythm, as illustrated in FIG. 5H.

Premature ventricular contraction (PVC) occurs when the ventricles fire an early impulse, causing the heart to beat earlier causing irregularity in the heart rhythm as illustrated in FIG. 51. Atrial fibrillation is a result of many sites within the atria firing electrical impulses in an irregular fashion causing irregular heart rhythm as illustrated in FIG. 5J.

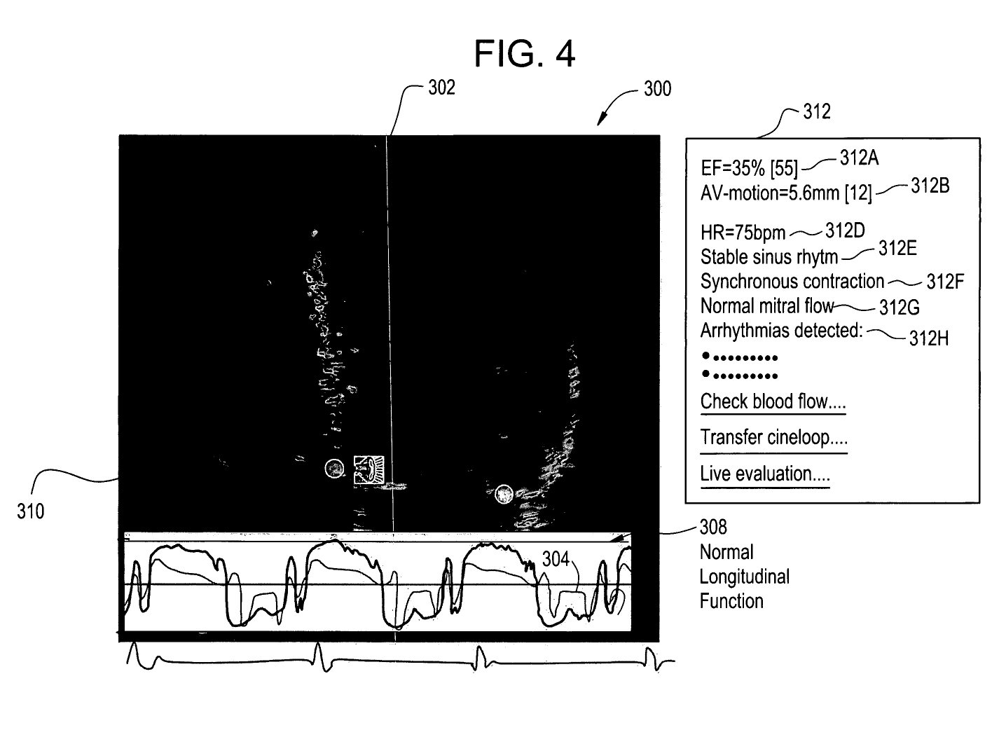

FIG. 4 illustrates one method for generating a clinical executive report, generally designated 300, using one or more methods discussed previously in accordance with one or more embodiments of the present invention. In at least one embodiment, one or more apical views of the heart are acquired. An AV-plane of the heart is identified, clinically relevant information is extracted and one or more clinically relevant reports are generated. In one or more embodiments, B-mode data 302 is displayed, although additional information may be gathered to identify the AV-plane, wherein such additional information may or may not displayed.

The localization's are, in one embodiment, provided in real-time, such that an erroneous location may be easily detected and a new location selected. Based at least in part, on this identification, a motion pattern 304 may be provided (in real-time for example), alone or with a graphical indication of normal ranges 306 and/or normal longitudinal functions 308 as provided in FIG. 4. Sound 310 associated with the location may be generated by the machine 5 or 105, enabling or assisting in rapid pattern recognition.

Based, at least in part on the clinically relevant information (velocity or strain rate profiles for example) extracted from the landmark locations may be assessed and a clinical executive report generated and displayed, alone or together with normal values and/or ranges (indicated in brackets). Such clinical executive report 312 may include one or more of the following parameters Ejection Fraction (EF) 312A, AV-motion 312B, Heart Rate (HR) 312D, sinus rhythm 312E, contractions 312F, mitral flow 312G, detected arrhythmias 312H (similar to those discussed previously with respect to FIGS. 5C-5J), etc.

One additional parameter that may be assessed and displayed in accordance with embodiment of the present invention comprises global function. In at least one embodiment, the present invention may determine if the global function is normal or reduced. It should be appreciated that Ejection Fraction or EF 312A, which indicates the proportion of blood pumped out of the heart with each beat, is a well-established parameter used in assessing global function. In the illustrated embodiment, the measured EF 312A is 35% where the normal value of 55% is indicated in brackets. In at least one embodiment, EF 312A is correlated with longitudinal motion of the AV-plane and may be indirectly assessed (as a rough estimate for example). Similarly, the longitudinal motion of the AV-plane 312B may be quantified and displayed, alone or together with normal values. In the illustrated embodiment, the measured longitudinal motion of the AV-plane is 5.6 mm, where the normal range of 12 mm is indicated in brackets.

One or more embodiments of the present invention may be used to determine if the patient is stable. The patient's heart rate (HR) 312D may be assessed directly from the periodicity in the velocity profile (without using an ECG for example). Hence, in one embodiment, heart-rate and variations in heart-rate may be displayed. Furthermore, embodiments may be used to determine whether the patient has normal sinus rhythm 312E (as discussed previously with respect to the FIGS. 5A and 5B) pand synchronous contraction 312F using temporal analysis of the extracted velocity and/or strain profiles (using the same or similar analysis techniques applied to ECG analysis in prior art for example).

It is also contemplated that one or more embodiments may be used to determine blood flow anomalies. The detected landmarks may be used to acquire necessary color flow and Doppler information that may be both visually assessed and quantified.

In one embodiment, a specialist (at a remote site for example) may be consulted to conduct an in-depth analysis of the acquired data. The ultrasound device (a hand-held device for example) may, in at least one embodiment, be adapted to communicate with such remote site or include a built-in communication device for downloading the acquired cineloops to the remote site.

Furthermore, live communications with the remote specialist may be established such that the remote specialist may see the acquired information in real-time, providing real-time audio-textual- or video-based feedback to the operator. For example, iMode or Wireless Application Protocols (alternatively referred to as “WAP”) used for mobile internet connection are suitable protocols for implementing such a live communication between the operator and the remote application specialist. While these protocols are discussed, other protocols are contemplated.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A method for generating an image responsive to moving cardiac structure and blood within a heart of a subject, the method comprising:
acquiring an apical view of the heart;
automatically identifying an AV-plane of the heart; and
generating a clinical executive report based, at least in part, on the AV-plane.

2. The method of claim 1 comprising using an ultrasound machine to acquire said apical view of the heart.

3. The method of claim 1 wherein said clinical executive report comprises at least one of the following parameters: Ejection Fraction, AV-motion, Heart Rate, sinus rhythm, contractions, mitral flow and detected arrhythmias.

4. The method of claim 1 comprising communicating the clinical executive report to at least one remote location.

5. The method of claim 4 comprising communicating the clinical executive report using a wireless application protocol.

6. In an ultrasound machine for generating an image responsive to moving cardiac structure and blood within a heart of a subject, a method comprising:
acquiring an apical view of the heart with the ultrasound machine;
generating an image of the apical view on a display of the ultrasound machine;
automatically identifying an AV-plane of the heart using said ultrasound machine; and
generating a clinical executive report using the ultrasound machine based on, at least in part, said identified AV-plane.

7. The method of claim 6 further comprising displaying said clinical executive report on a display of said ultrasound machine.

8. The method of claim 6 wherein the ultrasound machine comprises a hand-held device.

9. The method of claim. 6 wherein said clinical executive report comprises at least one of the following parameters: Ejection Fraction, AV-motion, Heart Rate, sinus rhythm, contractions, mitral flow and detected arrhythmias.

10. The method of claim 8 comprising communicating said clinical executive report using a wireless application protocol.

11. The method of claim 6 wherein automatically identifying an AV-plane comprises identifying at least one anatomical landmark.

12. The method of claim 11 wherein said at least one anatomical landmark comprises at least one of an apex of the heart and an AV-plane of the heart.

13. The method of claim 6 further comprising identifying at least one clinically relevant location using said AV-plane.

14. The method of claim 13 further comprising displaying indicia overlaying said AV-plane on the display of the ultrasound machine.

15. The method of claim 13 wherein the at least one clinically relevant location comprises at least one of lower parts of basal segments of the heart, lower parts of mid segments of the heart, at least one complete myocardial segment of the heart, at least one chamber of the heart, and at least one boundary between at least two chambers of the heart.

16. The method of claim 13 wherein said clinically relevant information comprises at least one of Doppler profile information, velocity profile information, strain rate profile information, strain profile information, M-mode information, deformation information, displacement information, and B-mode information.

17. In an ultrasound machine for generating an image responsive to moving cardiac structure and blood within a heart of a subject, an apparatus comprising:
a front-end arranged to transmit ultrasound waves into the moving cardiac structure and blood, generating received signals in response to ultrasound waves backscattered from the moving cardiac structure and blood;
at least one processor responsive to said received signals, acquiring an apical view of the heart with the ultrasound machine, generating an image of said apical view on a display of the ultrasound machine, automatically identifying an AV-plane of the heart using the ultrasound machine and generating a clinical executive report using the ultrasound machine based on, at least in part, said identified AV-plane.

18. The apparatus of claim 17 further comprising a display processor and monitor adapted to process generated position information and display indicia overlaying at least one of at least one anatomical landmark and at least one clinically relevant location.

19. The apparatus of claim 18 wherein said at least one clinically relevant location comprises at least one of lower parts of basal segments of the heart, lower parts of mid segments of the heart, at least one complete myocardial segment of the heart, at least one chamber of the heart, and at least one boundary between at least two chambers of the heart.

20. The apparatus of claim 18 wherein said at least one processor comprises at least one of a Doppler processor, a non-Doppler processor, a control processor, and a PC back-end.
